(19) United States
(12) Patent Application Publication
(10) Pub. No.: US 2014/0004086 A1
(43) Pub. Date: Jan. 2, 2014

(54) FORMATION OF CELL AGGREGATES

(71) Applicant: TISSUE GENESIS, INC., Honolulu, HI (US)

(72) Inventor: Hyun Joon Peak, Mililani, HI (US)

(73) Assignee: Tissue Genesis Inc., Honolulu, HI (US)

(21) Appl. No.: 13/843,814

(22) Filed: Mar. 15, 2013

Related U.S. Application Data
(60) Provisional application No. 61/666,660, filed on Jun. 29, 2012.

(51) Int. Cl. C12M 1/12; A61K 35/12; C12N 5/0775

(52) U.S. Cl. CPC C12M 25/04; C12N 5/0667; A61K 35/35
USPC 424/93.7; 435/297.1; 435/375; 435/377; 435/374; 435/347

(57) ABSTRACT

Cell aggregate forming chambers are described, suitable for automated loading and unloading, where the airtight chamber contains a mold with a plurality of cavities, where there is an inlet and an outlet for cells, and where air is filtered before it comes into the chamber. Method of using the chamber include injecting cells into the chamber, providing conditions where the cells may grow to form cell aggregates, and extracting the cell aggregates through a cell outlet.

### PRIORITY CLAIM

This application claims the benefit of provisional patent application Ser. No. 61/666,680, filed Jun. 29, 2012, titled “Formation of Cell Aggregates”, the contents which are incorporated herein by reference in their entirety

### TECHNICAL FIELD

The invention relates to a system and method for forming cell aggregates.

### BACKGROUND

Cell aggregates may be formed in various ways. For example, in Tekin et al., Stimuli-responsive microwells for formation and retrieval of cell aggregates, Lab Chip 2010, 10(18):2411-8, aggregates are formed in lithographically-created microwells. Similar microwells are described in Choi et al., Controlled-size embryoid body formation in concave microwell arrays, Biomaterials 2010, 31:4296-4303.

One problem with existing chambers for creating cell aggregates is that the chambers may not be suitable for automation. Moreover, maintaining sterility and isolation from the environment is a continuing problem.

### BRIEF SUMMARY

Described herein are various inventions, particular examples of which are summarized here. In one embodiment, a cell aggregate forming chamber comprises: at least one cell inlet; at least one cell outlet; an air inlet separated from outside air through a filter sized to exclude biological organisms; a mold of non-cell adherent material, comprising a plurality of cavities; and a transparent cover over the mold, so as to provide an airtight space between the cover and the mold.

In another embodiment, a method for forming cell aggregates comprises:

providing the chamber described above; (b) injecting isolated cells into the chamber through one of the one or more cell inlets; (c) providing conditions within the chamber conducive to cell growth, thereby forming cell aggregates; and (d) extracting the cell aggregates through one of the one or more cell outlets.

Various additional embodiments, including additions and modifications to the above embodiments, are described herein.

### BRIEF DESCRIPTION OF THE DRAWINGS

The accompanying drawings, which are incorporated into this specification, illustrate one or more exemplary embodiments of the inventions disclosed herein and, together with the detailed description, serve to explain the principles and exemplary implementations of these inventions. One of skill in the art will understand that the drawings are illustrative only, and that what is depicted therein may be adapted based on the text of the specification or the common knowledge within this field.

In the drawings:

FIG. 1 is a drawing of an example aggregate forming chamber.

FIG. 2 is a top view of an example aggregate forming chamber.

FIG. 3 is a cross-sectional view of an example aggregate forming chamber.

### DETAILED DESCRIPTION

Various example embodiments of the present inventions are described herein in the context of forming cell aggregates.

Those of ordinary skill in the art will realize that the following detailed description is illustrative only and is not intended to be in any way limiting. Other embodiments will readily suggest themselves to such skilled persons having the benefit of this disclosure. Reference will now be made in detail to implementations as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application, safety, regulatory, and business constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

### Formation of Aggregates

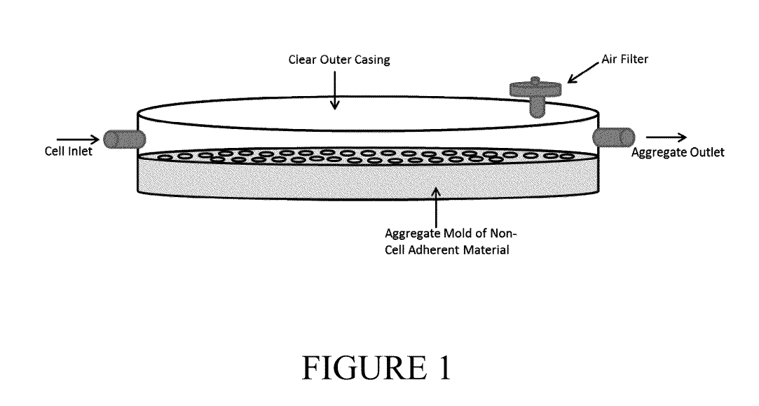

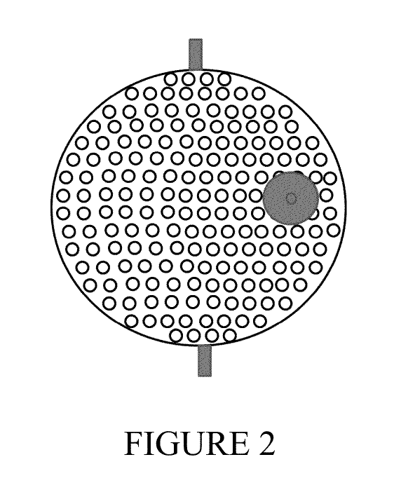

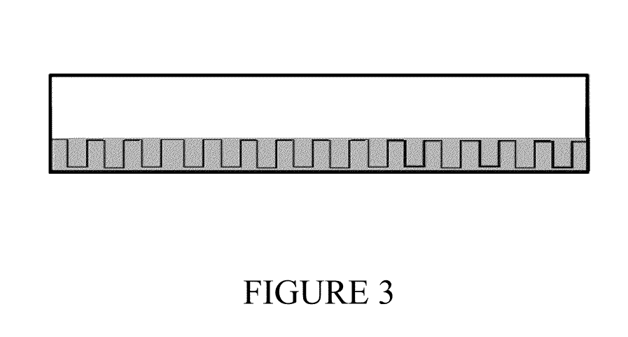

In one embodiment of the present disclosure, following automated or manual cell isolation, freshly isolated cells of any type may be directly transferred to an aggregate forming chamber such as that shown in FIGS. 1-3. Cultured cells may be placed in the chamber to form aggregates of uniform size. The chamber may contain one or more inlets and one or more outlets. Preferably, the chamber has an air filter. Preferably, the aggregate mold is made of non-cell-adherent material, and contains holes or cavities as shown. The holes or cavities are preferably cylindrical or hemispherical. The chamber may in one embodiment be formed with a clear outer casing. The use of a clear casing makes it possible to inspect the growing cell aggregates without breaking sterility.

The aggregate forming chamber may be easily incorporated into a disposable unit or cartridge, for use in an automated system. In various embodiments, this automated system may also digest tissue and/or isolate cells, such as adipose cells obtained from liposuction or other surgery.

In the aggregate forming chamber, spherical aggregates may be allowed to spontaneously form by viable/healthy cells, separating out most of apoptotic and necrotic cells in the inlet product. This system has a number of advantages. For example, it may eliminate negative effects posed by apoptotic and necrotic cells in the product. It may also provide a biomimicking 3-D environment for any types of cells. Further, it may allow accelerated recovery of cells immediately following collagenase treatment.

Following formation, the chamber can be inverted and shaken lightly to allow aggregates exit out of the holes in the mold and be collected via a syringe through an outlet. Aggregates can be further cultured within the same chamber for various applications.

The use of uniform spherical aggregates may be advantageous over aggregates of random size. For example, size restriction and uniformity prevents necrosis of cells in the core. Also, uniform size of aggregates may allow convenient dosage calculation. Further, uniform size may allow ease of identification and delivery.

The described system allows for ease of tissue construct formation with stem cells. Aggregates can be formed with undifferentiated and differentiated stem cells from various origin (bone marrow, adipose, skin, muscle, heart, nerve, etc), and these aggregates can be used as a building block and assembled together to form a three-dimensional tissue construct with and without a scaffold. Conventional in-vitro culture and differentiation of stem cells may be carried out in a 2-D culture. To fabricate a tissue construct, these differentiated cells should preferably be collected via trypsinization and seeded onto a scaffold material. During this process, some of the differentiated cells are not expected to survive and hence the cell seeding efficiency is expected to be decreased. These cells also may take a substantial amount of time to attach to the surface, occupy and fill up the void space within a construct. Following formation of cell aggregates, they can be induced to differentiate in a 3-D environment within the tissue mold and seeded onto a scaffold material. By eliminating trypsinization step and reducing the time to fill the void space, a tissue construct can be rapidly fabricated without affecting cell seeding efficiency and survival rate. In case of allogeneic or xenogenic cells, aggregates can also be immunoisolated by encapsulating in various hydrogel microsphere prior to administration.

In one embodiment, cell aggregates can be cryopreserved. Compared to individual cells in suspension, cell aggregates can be expected to improve cell survival and maintain their function during and following cryopreservation.

### Stromal Vascular Fraction Cell Aggregate-Based Microtissue (SAM)

Stromal vascular fraction cell Aggregate-based Microtissue (“SAM”) is described herein as an embodiment. SAM may be advantageous over the typical use of stromal vascular fraction (“SVF”) cells. For example, SVC cell survival may be improved, after isolation. There may be accelerated and improved separation of apoptotic and necrotic cells from healthy/viable cells. The maintenance of pluripotency of stem cells within SVF cells may be improved. The maintenance/stabilization of phenotypes following induced differentiation may be improved. The secretion of growth factors, cytokines, and other proteinaceous materials may be improved. Abnormal and unintended growth of cells (abnormal gene expression and ploidity, hypertrophy, etc.) may be prevented. Cellular organization (vascularization, spatial organization, etc) may also be improved.

In one embodiment, adipose-derived stromal vascular fraction (SVF) cells aggregates can be mixed with adipose tissue for fat grafting. For conventional SVF cell-assisted fat grafting, adipose tissue may be mixed with either SVF cells in suspension or in a pellet. Retention of individual cells in suspension is expected to be poor because cells can leave the implant site as the excess fluid recedes from the graft. When cell pellet is mixed with adipose tissue, an exact dosage of the cells per unit volume of fat graft may be unclear and inconsistent. By mixing SAMs with adipose tissue, cell aggregates can be trapped within the fat graft more effectively and consequently improve their retention within the graft. Mixing a unit volume of adipose tissue with a predetermined number of SVF cell aggregates may allow a delivery of a consistent dosage throughout multiple graft injections during the procedure. SAMs can also contain microvasculatures within the aggregates, which can facilitate accelerated incorporation of SAMs into the implant area and improved graft survival. SAMs secreted increased amount of growth factors and cytokines compared to individual SVF cells, which can also improve graft survival and incorporation.

In one embodiment, SAMs can be injected by themselves or along with a filler for aesthetic and other medical procedures for skin.

What is claimed is:

1. A cell aggregate forming chamber comprising:
at least one cell inlet;
at least one cell outlet;
an air inlet separated from outside air through a filter sized to exclude biological organisms;
a mold of non-cell adherent material, comprising a plurality of cavities; and
a transparent cover over the mold, so as to provide an airtight space between the cover and the mold.

2. The cell aggregate forming chamber of claim 1, wherein the cavities are cylinders;

3. The cell aggregate forming chamber of claim 1, further comprising:
a cell inlet valve in communication with the cell inlet;
a cell digestion chamber upstream of the cell inlet valve; and
a centrifuge upstream of the cell inlet valve.

4. A method for forming cell aggregates comprising:
(a) providing the chamber of claim 1;
(b) injecting isolated cells into the chamber through one of the one or more cell inlets;
(c) providing conditions within the chamber conducive to cell growth, thereby forming cell aggregates; and
(d) extracting the cell aggregates through one of the one or more cell outlets.

5. The method of claim 4, further comprising, after step (c) and before step (d):
inverting the chamber; and
shaking the chamber.

6. The method of claim 4, wherein step (d) is carried out by extracting the cell aggregates through one of the one or more cell outlets using a syringe.

7. The method of claim 4, wherein the isolated cells comprise stem cells, and further comprising, after step (c) and before step (d):
further culturing cells within the cell aggregates to promote cell differentiation.

8. The method of claim 4, further comprising:
seeding the cell aggregates are seeded into a three dimensional scaffold.

9. The method of claim 4, further comprising:
encapsulating the cell aggregates in a water-permeable membrane.

10. The method of claim 4, further comprising:
cryopreserving the cell aggregates.

11. The method of claim 4, wherein the isolated cells are stromal vascular fraction cells.

12. The method of claim 11, further comprising:
mixing the cell aggregates with adipose tissue.

13. The method of claim 12, further comprising:
grafting said mixture into a patient.
